(19) United States
(12) Patent Application Publication
(10) Pub. No.: US 2014/0336723 A1
(43) Pub. Date: Nov. 13, 2014

(54) ACTIVE GLASSES FOR OPTIC NERVE STIMULATION

(71) Applicants: Omry Ben-Ezra, Tel-Aviv (IL); Viljem Fuchs, Dol pri Ljubljani (SI); Andrej Tomeljak, Ljubljani (SI); Ami Dror, Tel-Aviv (IL); Todd Mattingly, Bentonville, AR (US)

(72) Inventors: Omry Ben-Ezra, Tel-Aviv (IL); Viljem Fuchs, Dol pri Ljubljani (SI); Andrej Tomeljak, Ljubljani (SI); Ami Dror, Tel-Aviv (IL); Todd Mattingly, Bentonville, AR (US)

(21) Appl. No.: 14/359,137

(22) PCT Filed: Oct. 15, 2012
(86) PCT No.: PCT/US2012/060190

§ 371 Date: May 19, 2014

Related U.S. Application Data
(60) Provisional application No. 61/561,416, filed on Nov. 18, 2011.

(51) Int. Cl. A61N 1/36; A61N 1/04

(52) U.S. Cl. CPC A61N 1/36046; A61N 1/36025; A61N 1/0484
USPC 607/45; 607/53

(57) ABSTRACT

Active glasses for stimulating optic nerves of a user.

### CROSS-REFERENCE TO RELATED APPLICATIONS

This application claims the benefit of the filing date of U.S. provisional patent application Ser. No. 61/561,416, filed on Nov. 18, 2011, attorney docket number 092847.001306, the disclosure of which is incorporated herein by reference.

### BACKGROUND

This disclosure relates to active glasses for stimulating optic nerves of a user.

### BRIEF DESCRIPTION OF THE DRAWINGS

FIG. 1 is an illustration of an exemplary embodiment of a system for stimulating optic nerves.

FIGS. 2A-2F is a flow chart of an exemplary embodiment of a method for operating the system of FIG. 1.

FIGS. 3A-3C are flow charts of exemplary embodiments of methods for operating the system of FIG. 1.

FIG. 4 is a top view of an exemplary embodiment of active glasses.

FIG. 5 is a rear view of the active glasses of FIG. 4.

FIG. 6 is a bottom view of the active glasses of FIG. 4.

FIG. 7 is a front view of the active glasses of FIG. 4.

FIG. 8 is a perspective view of the active glasses of FIG. 4.

FIG. 9 is a side view of the active glasses of FIG. 4.

FIGS. 10A-10B are perspective views of an exemplary embodiment of active glasses and a prescription attachment.

### DETAILED DESCRIPTION

In the drawings and description that follows, like parts are marked throughout the specification and drawings with the same reference numerals, respectively. The drawings are not necessarily to scale. Certain features of the invention may be shown exaggerated in scale or in somewhat schematic form and some details of conventional elements may not be shown in the interest of clarity and conciseness. The present invention is susceptible to embodiments of different forms. Specific embodiments are described in detail and are shown in the drawings, with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results. The various characteristics mentioned above, as well as other features and characteristics described in more detail below, will be readily apparent to those skilled in the art upon reading the following detailed description of the embodiments, and by referring to the accompanying drawings.

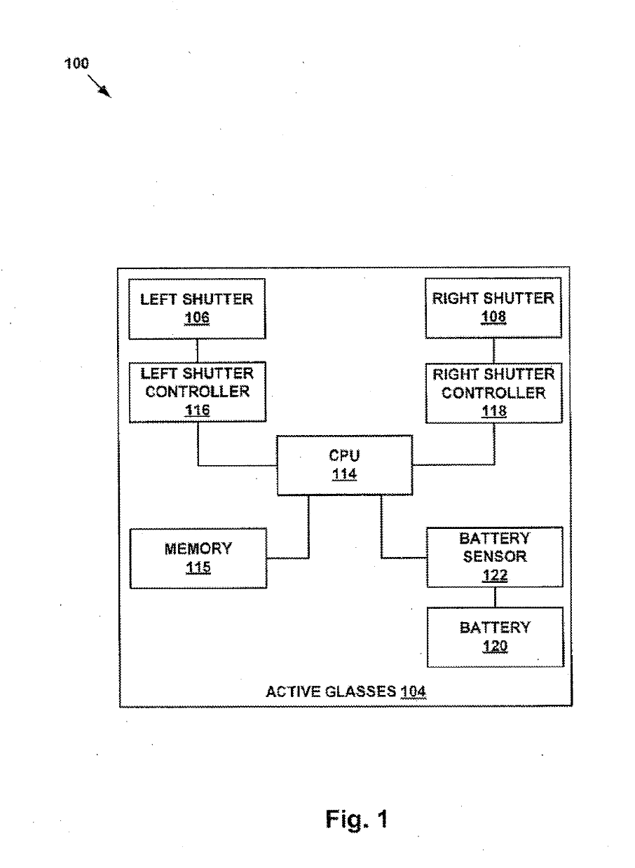

Referring initially to FIG. 1, a system 100 for stimulating optic nerves includes a pair of active glasses 104 having a left shutter 106 and a right shutter 108. In an exemplary embodiment, the active glasses 104 include a frame and the shutters, 106 and 108, are provided as left and right viewing lenses mounted and supported within the frame.

In an exemplary embodiment, the shutters, 106 and 108, are liquid crystal cells that open when the cell goes from opaque to clear, and the cell closes when the cell goes from clear back to opaque. In an exemplary embodiment, the user of the active glasses 104 may be able to see ambient light when the liquid crystal cells of the shutters, 106 and/or 108, of the active glasses 104 become 25-30 percent transmissive. Thus, the liquid crystal cells of a shutter, 106 and/or 108, is considered to be open when the liquid crystal cell becomes 25-30 percent transmissive. The liquid crystal cells of a shutter, 106 and/or 108, may also transmit more than 25-30 percent of light when the liquid crystal cell is open.

In an exemplary embodiment, the shutters, 106 and 108, of the active glasses 104 include liquid crystal cells having a PI-cell configuration utilizing a low viscosity, high index of refraction liquid crystal material such as, for example, Merck MLC6080. In an exemplary embodiment, the PI-cell thickness is adjusted so that in its relaxed state it forms a ½-wave retarder. In an exemplary embodiment, the PI-cell is made thicker so that the ½-wave state is achieved at less than full relaxation. One of the suitable liquid crystal materials is MLC6080 made by Merck, but any liquid crystal with a sufficiently high optical anisotropy, low rotational viscosity and/or birefringence may be used. The shutters, 106 and 108, of the active glasses 104 may also use a small cell gap, including, for example, a gap of 4 microns. Furthermore, a liquid crystal with a sufficiently high index of refraction and low viscosity may also be suitable for use in the shutters, 106 and 108, of the active glasses 104.

In an exemplary embodiment, the Pi-cells of the shutters, 106 and 108, of the active glasses 104 work on an electrically controlled birefringence (“ECB”) principle. Birefringence means that the Pi-cell has different refractive indices, when no voltage or a small catching voltage is applied, for light with polarization parallel to the long dimension of the Pi-cell molecules and for light with polarization perpendicular to long dimension, no and ne. The difference no−ne=Δn is optical anisotropy. Δn×d, where d is thickness of the cell, is optical thickness. When Δn×d=½λ the Pi-cell is acting as a ½ wave retarder when cell is placed at 45° to the axis of the polarizer. So optical thickness is important not just thickness. In an exemplary embodiment, the Pi-cells of the shutters, 106 and 108, of the active glasses 104 are made optically too thick, meaning that Δn×d>½λ. The higher optical anisotropy means thinner cell—faster cell relaxation. In an exemplary embodiment, when voltage is applied the molecules' of the Pi-cells of the shutters, 106 and 108, of the active glasses 104 long axes are perpendicular to substrates—homeotropic alignment, so there is no birefringence in that state, and, because the polarizers have transmitting axes crossed, no light is transmitted. In an exemplary embodiment, Pi-cells with polarizers crossed are said to work in normally white mode and transmit light when no voltage is applied. Pi-cells with polarizers' transmitting axes oriented parallel to each other work in a normally black mode, i.e., they transmit light when a voltage is applied.

In an exemplary embodiment, when high voltage is removed from the Pi-cells, the opening of the shutters, 106 and/or 108, start. This is a relaxation process, meaning that liquid crystal (“LC”) molecules in the Pi-cell go back to the equilibrium state, i.e. molecules align with the alignment layer, i.e. the rubbing direction of the substrates. The Pi-cell's relaxation time depends on the cell thickness and rotational viscosity of the fluid.

In general, the thinner the Pi-cell, the faster the relaxation. In an exemplary embodiment, the important parameter is not the Pi-cell gap, d, itself, but rather the product Δnd, where Δn is the birefringence of the LC fluid. In an exemplary embodiment, in order to provide the maximum light transmission in its open state, the head-on optical retardation of the Pi-cell, Δnd, should be λ/2. Higher birefringence allows for thinner cell and so faster cell relaxation. In order to provide the fastest possible switching fluids with low rotational viscosity and higher birefringence—Δn (such as MLC 6080 by EM industries) are used.

In an exemplary embodiment, in addition to using switching fluids with low rotational viscosity and higher birefringence in the Pi-cells, to achieve faster switching from opaque to clear state, the Pi-cells are made optically too thick so that the ½-wave state is achieved at less than full relaxation. Normally, the Pi-cell thickness is adjusted so that in its relaxed state it forms a ½-wave retarder. However, making the Pi-cells optically too thick so that the ½-wave state is achieved at less than full relaxation results in faster switching from opaque to clear state. In this manner, the shutters 106 and 108 of the exemplary embodiments provide enhanced speed in opening versus prior art LC shutter devices that, in an exemplary experimental embodiment, provided unexpected results.

In an exemplary embodiment, a catch voltage may then be used to stop the rotation of the LC molecules in the Pi-cell before they rotate too far. By stopping the rotation of the LC molecules in the Pi-cell in this manner, the light transmission is held at or near its peak value.

In an exemplary embodiment, one or more of the shutters 106 and 108 may, in the alternative, use twisted nematic (“TN”) liquid crystal cells. The general design and operation of TN cells is considered well known in the art.

In an exemplary embodiment, one or more of the shutters 106 and 108 may, in the alternative, use other types of liquid crystal cells. The general design and operation of liquid crystal cells, in general, is considered well known in the art.

In an exemplary embodiment, the active glasses 104 have a central processing unit (“CPU”) 114. The CPU 114 may, for example, include a general purpose programmable controller, an application specific intergrated circuit (“ASIC”), an analog controller, a localized controller, a distributed controller, a programmable state controller, and/or one or more combinations of the aforementioned devices.

The CPU 114 is operably coupled to a left shutter controller 116 and a right shutter controller 118 for monitoring and controlling the operation of the shutter controllers. In an exemplary embodiment, the left and right shutter controllers, 116 and 118, are in turn operably coupled to the left and right shutters, 106 and 108, of the active glasses 104 for monitoring and controlling the operation of the left and right shutters. The shutter controllers, 116 and 118, may, for example, include a general purpose programmable controller, an ASIC, an analog controller, an analog or digital switch, a localized controller, a distributed controller, a programmable state controller, and/or one or more combinations of the aforementioned devices.

A battery 120 is operably coupled to at least the CPU 114 and provides power for operating one or more of the CPU, and the shutter controllers, 116 and 118, of the active glasses 104. A battery sensor 122 is operably coupled to the CPU 114 and the batter 120 for monitoring the amount of power remaining in the battery.

In an exemplary embodiment, the CPU 114 may monitor and/or control the operation of one or more of the shutter controllers, 116 and 118, and the battery sensor 122. Alternatively, or in addition, one or more of the shutter controllers, 116 and 118, and the battery sensor 122 may include a separate dedicated controller and/or a plurality of controllers, which may or may not also monitor and/or control one or more of the shutter controllers, 116 and 118, and the battery sensor 122. Alternatively, or in addition, the operation of the CPU 114 may at least be partially distributed among one or more of the other elements of the active glasses 104.

In an exemplary embodiment, the CPU 114, the shutter controllers, 116 and 118, the battery 120, and the battery sensor 122 are mounted and supported within the frame of the active glasses 104. In an exemplary embodiment, during operation of the system 100, the CPU 114 controls the operation of the shutters, 106 and 108, of the active glasses 104 as a function of a therapy sequence stored in memory 115 operably connected to the CPU 114. In an exemplary embodiment, a therapy sequence defines a sequence for opening and closing the shutters, 106 and 108, to stimulate the visual system of a user of the active glasses 104. Stimulation of the visual system of the user may include utilizing visual properties such as enhanced contrast, dark adaptation, neighbor cell inhibition, pupil size modulation, and blink modulation. In an exemplary embodiment, the CPU 114 may direct the left shutter controller 116 to open the left shutter 106 and/or direct the right shutter controller 118 to open the right shutter 108.

In an exemplary embodiment, the shutter controllers, 116 and 118, control the operation of the shutters, 106 and 108, respectively, by applying a voltage across the liquid crystal cells of the shutter. In an exemplary embodiment, the voltage applied across the liquid crystal cells of the shutters, 106 and 108, alternates between negative and positive. In an exemplary embodiment, the liquid crystal cells of the shutters, 106 and 108, open and close the same way regardless of whether the applied voltage is positive or negative. Alternating the applied voltage prevents the material of the liquid crystal cells of the shutters, 106 and 108, from plating out on the surfaces of the cells.

In an exemplary embodiment, each of the shutters, 106 and 108, may be divided into shutter regions that may be independently controlled by the shutter controllers, 116 and 118, respectively. In this case, a specific region of the user's visual field may be affected using the shutter regions. In an exemplary embodiment, each of the shutters, 106 and 108, and/or shutter regions may be configured with different polarization orientations. For example, each of the shutters, 106 and 108, and/or shutter regions may be linearly polarized at a different angle.

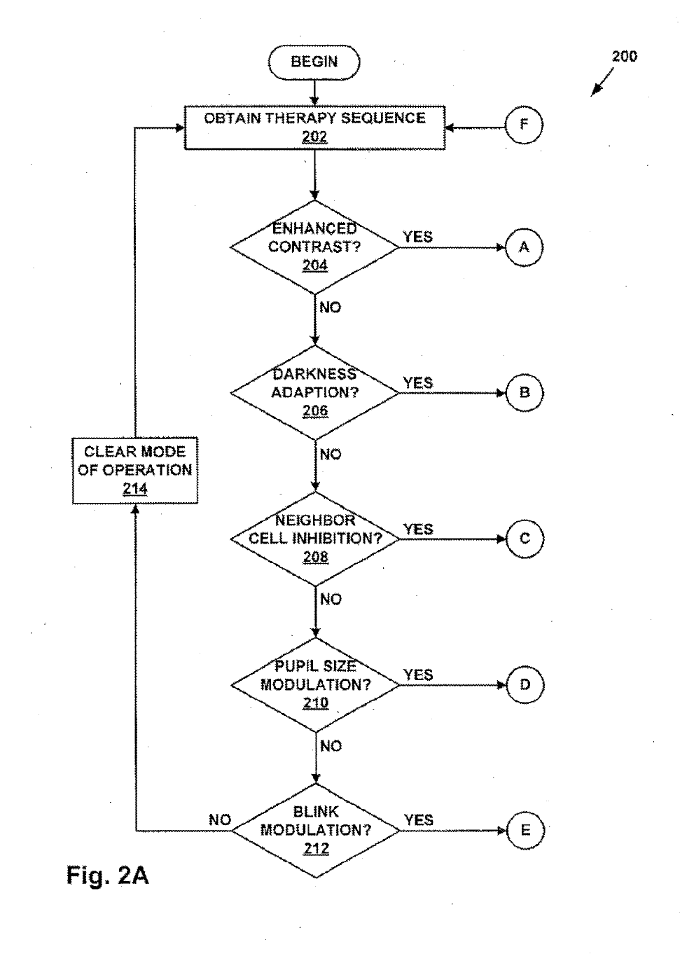

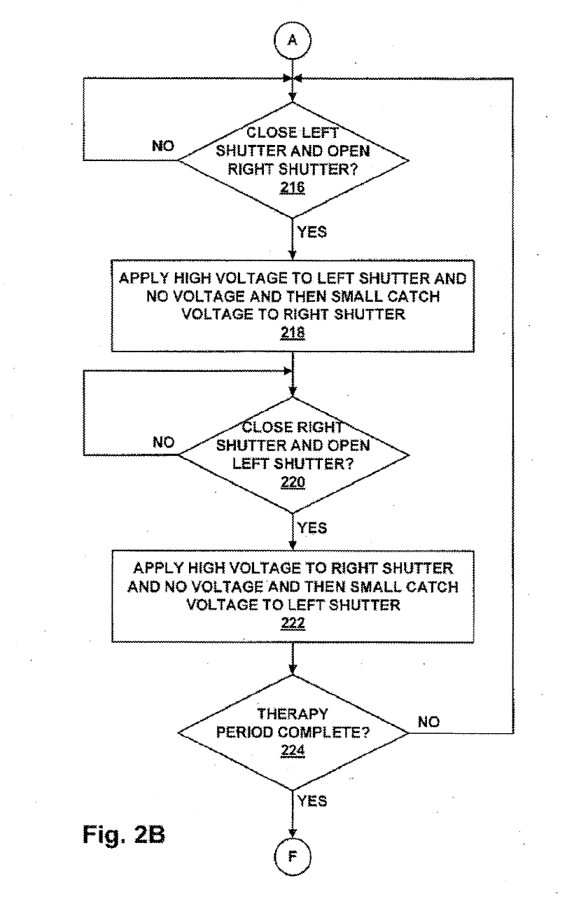

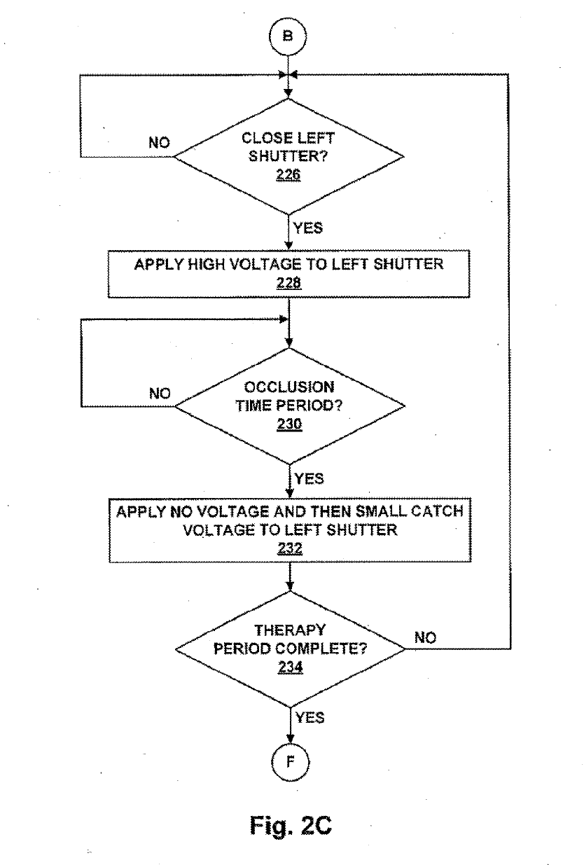

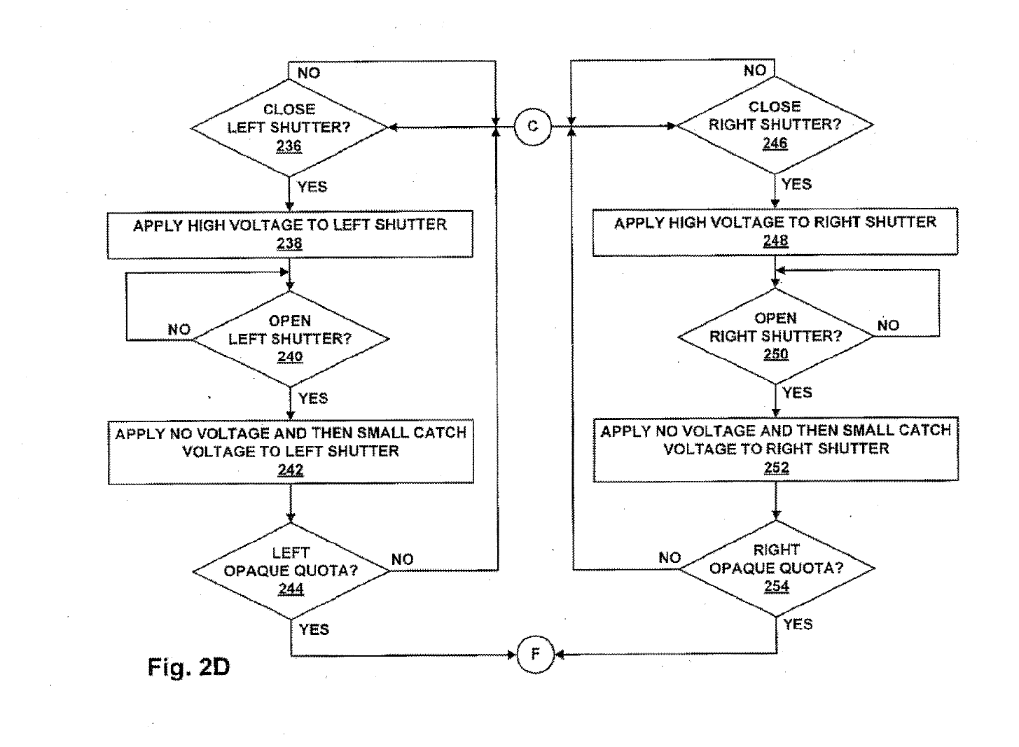

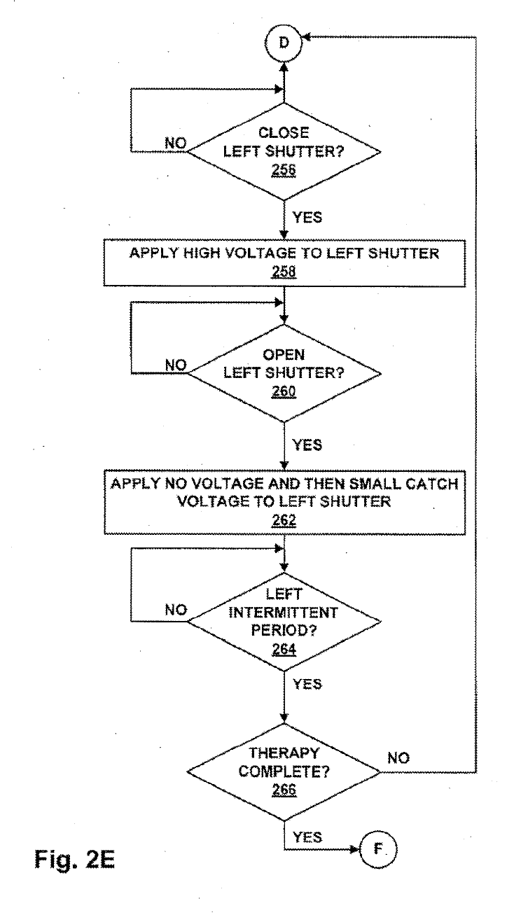

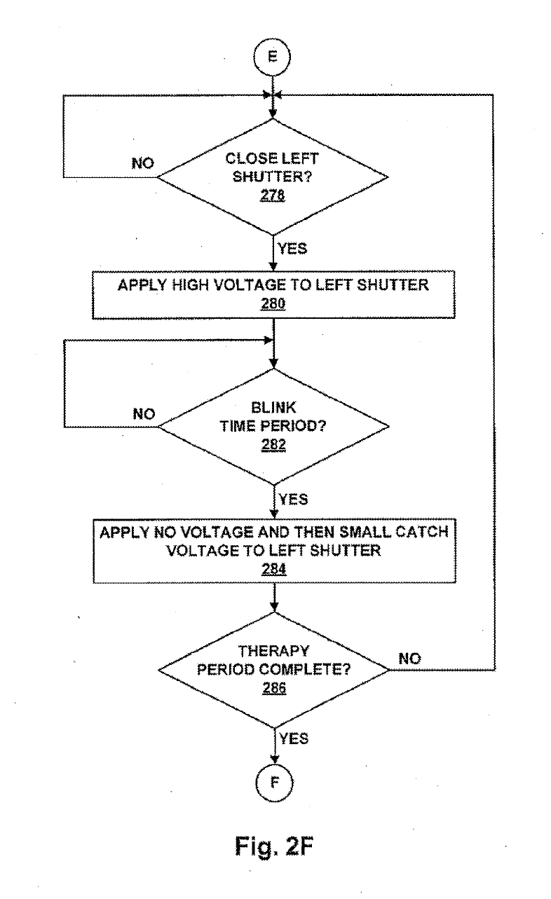

In an exemplary embodiment, during operation of the system 100, as illustrated in FIGS. 2A-2F, the system may implement an optic nerve stimulation method 200 in which a therapy sequence is obtained in 202. The therapy sequence may define a sequence of opened and closed states for each of the left shutter 106 and right shutter 108. In an exemplary embodiment, the therapy sequence is configured to utilize one or more visual properties of a visual system of a user such as enhanced contrast, dark adaptation, neighbor cell inhibition, pupil size modulation, and blink modulation.

In FIG. 2A, even though each visual property is shown as being stimulated in a separate flow (e.g., enhanced contrast 204, dark adaptation 206, neighbor cell inhibition 208, pupil size modulation 210, and blink modulation 212) of the method 200, each flow may be configured to utilize multiple visual properties. In other words, each of the separate flows of the method 200 may be predominantly configured to utilize the shown visual property while secondarily utilizing additional visual properties.

If in 204 it is determined that the therapy sequence is configured to predominantly stimulate enhanced contrast, the process proceeds to 216. In an exemplary embodiment, by alternating the active glasses 104 from transparent to opaque, the eye and nerve behind the corresponding lens shutter, 106 or 108, goes from full illumination to relative darkness within milliseconds. Occlusion is typically applied to one eye only, to avoid significant occlusion of vision. In an exemplary embodiment, the lens shutter, 106 or 108, is transparent for longer than 0.1 seconds. In this case, the constant flickering maintains a “first sight” effect, where visual stimuli are more clearly registered at first sight rather than at a constant stare.

If in 216 it is determined that the left shutter 106 will be closed and the right shutter 108 will be opened, then in 218, a high voltage is applied to the left shutter 106 and no voltage followed by a small catch voltage are applied to the right shutter 108 by the shutter controllers, 116 and 118, respectively. In an exemplary embodiment, applying the high voltage to the left shutter 106 closes the left shutter, and applying no voltage to the right shutter 108 starts opening the right shutter. In an exemplary embodiment, the subsequent application of the small catch voltage to the right shutter 108 prevents the liquid crystals in the right shutter from rotating too far during the opening of the right shutter 108. As a result, in 218, the left shutter 106 is closed and the right shutter 108 is opened.

If in 220 it is determined that the left shutter 106 will be opened and the right shutter 108 will be closed, then in 222, a high voltage is applied to the right shutter 108 and no voltage followed by a small catch voltage are applied to the left shutter 106 by the shutter controllers, 118 and 116, respectively. In an exemplary embodiment, applying the high voltage to the right shutter 108 closes the right shutter, and applying no voltage to the left shutter 106 starts opening the left shutter. In an exemplary embodiment, the subsequent application of the small catch voltage to the left shutter 106 prevents the liquid crystals in the left shutter from rotating too far during the opening of the left shutter 106. As a result, in 222, the left shutter 106 is opened and the right shutter 108 is closed.

In an exemplary embodiment, the magnitude of the catch voltage used in 218 and 222 ranges from about 10 to 20% of the magnitude of the high voltage used in 218 and 222.

In an exemplary embodiment, during the operation of the system 100, the CPU 114 will direct each shutter, 106 and 108, to alternatively open and close until the therapy period is determined to be complete in 224. The CPU 114 may have an internal timer to maintain proper shutter sequencing.

In an exemplary embodiment, the combination of viscous liquid crystal material and narrow cell gap in the shutters, 106 and 108, may result in a cell that is optically too thick. The liquid crystal in the shutters, 106 and 108, blocks light transmission when voltage is applied. Upon removing the applied voltage, the molecules in the liquid crystals in the shutters, 106 and 108, rotate back to the orientation of the alignment layer. The alignment layer orients the molecules in the liquid crystal cells to allow light transmission. In a liquid crystal cell that is optically too thick, the liquid crystal molecules rotate rapidly upon removal of power and thus rapidly increase light transmission but then the molecules rotate too far and light transmission decreases. The time from when the rotation of the liquid crystal cell molecules starts until the light transmission stabilizes, i.e. liquid crystal molecules rotation stops, is the true switching time.

In an exemplary embodiment, when the shutter controllers, 116 and 118, apply the small catch voltage to the shutters, 106 and 108, this catch voltage stops the rotation of the liquid crystal cells in the shutters before they rotate too far. By stopping the rotation of the molecules in the liquid crystal cells in the shutters, 106 and 108, before they rotate too far, the light transmission through the molecules in the liquid crystal cells in the shutters is held at or near its peak value. Thus, the effective switching time is from when the liquid crystal cells in the shutters, 106 and 108, start their rotation until the rotation of the molecules in the liquid crystal cells is stopped at or near the point of peak light transmission.

If in 206 it is determined that the therapy sequence is configured to predominantly stimulate darkness adaptation, the process proceeds to 226. The sensitivity of each photoreceptor in the human eye is inversely proportional to the light intensity over time. For example, at times of very dim illumination, each photoreceptor cell adapts to the darkness thereby rapidly increasing the sensitivity of the cell to light. The adaptation function behaves exponentially over time, making the cells approximately 50 times more sensitive to light within the first two minutes. By occluding an eye with a shutter, 106 or 108, to cause relative darkness, the cells of the occluded eye adapt to the darkness to increase their sensitivity to light. When the shutter, 106 or 108, covering the eye is returned to the transparent mode, the now over sensitive eye will react in excess to the ambient light if at least 30 seconds of occlusion were previously applied.

If in 226 it is determined that the left shutter 106 will be closed, then in 228, a high voltage is applied to the left shutter 106 by the left shutter controller 116. In an exemplary embodiment, applying, the high voltage to the left shutter 106 closes the left shutter. In an exemplary embodiment, the right shutter 108 is already opened; thus, in 218, the left shutter 106 is closed and the right shutter 108 remains open.

If in 230 it is determined that the occlusion time period has passed, then in 232, no voltage followed by a small catch voltage is applied to the left shutter 106 by the left shutter controller 116. For example, the occlusion time period for the left shutter 106 may be at least 30 seconds to allow the left eye of the user to become over sensitive to ambient light. In an exemplary embodiment, applying no voltage to the left shutter 106 starts opening the left shutter. In an exemplary embodiment, the subsequent application of the small catch voltage to the left shutter 106 prevents the liquid crystals in the left shutter from rotating too far during the opening of the left shutter 106. As a result, in 232, the left shutter 106 is opened and the right shutter 108 remains open.

Alternatively, the right shutter 108 may be controlled by the right shutter controller 118 as discussed above in 226-232. In this case, the right shutter 108 may be closed for the occlusion time period and then opened while the left shutter 106 remains open.

If in 208 it is determined that the therapy sequence is configured to predominantly minimize neighbor cell inhibition, the process proceeds to 236. Each retinal cell of the user, when activated, produces inhibition of neighboring cells. The inhibition of neighboring cells is a natural mechanism that may facilitate the perception of boundaries and straight lines. Typically, the inhibition is not long lasting (i.e., rapidly decays within minutes) and takes time to accumulate. By using the shutters, 106 and 108, for timed occlusions, it is possible to avoid the buildup of such inhibitions by allowing for the effect to be cleared. In an exemplary embodiment, each eye of the user is occluded by the respective shutter, 106 and 108, for approximately 20 seconds of every time period (e.g., 1 minute, 2 minutes, etc.), thus not allowing for full inhibition build up and periodic clearing of any such effect. In an exemplary embodiment, each of left shutter 106 and the right shutter 108 may be controlled independently (i.e., in parallel) as discussed below to stimulate inhibition of neighboring cells.

If in 236 it is determined that the left shutter 106 will be closed, then in 238, a high voltage is applied to the left shutter 106 by the shutter controller 116. In an exemplary embodiment, applying the high voltage to the left shutter 106 closes the left shutter. If in 240 it is determined that the left shutter 106 will be opened, then in 242, no voltage followed by a small catch voltage is applied to the left shutter 106 by the shutter controllers 116. In an exemplary embodiment, applying no voltage to the left shutter 106 starts opening the left shutter. In an exemplary embodiment, the subsequent application of the small catch voltage to the left shutter 106 prevents the liquid crystals in the left shutter from rotating too far during the opening of the left shutter 106. In an exemplary embodiment, the activation pattern of the left shutter 106 may be at low frequencies (below about two Hz) or at high frequencies (above about 50 Hz).

If in 244 if is determined that the left opaque quota is satisfied, then the process proceeds to 202. The left opaque quota may specify that the left shutter 106 should be closed (i.e., opaque) for a predetermined portion of a time period. For example, the left opaque quota may specify that the left shutter 106 should be closed for about 20 seconds of every one minute time period.

In 246-254, the right shutter 108 is controlled by the right shutter controller 118 in a substantially similar manner as discussed above for the left shutter 106 in 236-244. In this case, the right shutter 108 is activated to stimulate neighbor cell inhibition until a right opaque quota is satisfied in 254. The right opaque quota may be different from the left opaque quota; however, the time period for activation is typically the same for the left shutter 106 and right shutter 108 so that their respective quotas are satisfied at approximately the same time before the process returns to 202.

If in 210 it is determined that the therapy sequence is configured to predominantly stimulate pupil size modulation, the process proceeds to 256. the size of the pupil is controlled by a complex reflex arch, incorporating inputs from the eye, the contra lateral eye and the autonomic nerve system. Thus, changes in the light intensity affect both eyes. By applying intermittent shuttering over an eye, changes in momentary lighting can be achieved and the relation between pupil size and the average ambient light may be modulated to benefit the patient. It is further acknowledged that the pupils are faster to constrict than to relax, thus rapid transitions between two states of light exposure will result with a pupil size that is more constricted.

If in 256 it is determined that the left shutter 106 will be closed, then in 258, a high voltage is applied to the left shutter 106 by the left shutter controller 116. In an exemplary embodiment, applying the high voltage to the left shutter 106 closes the left shutter. If in 260 it is determined that the left shutter 106 will be opened, then in 262, no voltage followed by a small catch voltage is applied to the left shutter 106 by the left shutter controller 116. In an exemplary embodiment, applying no voltage to the left shutter 106 starts opening the left shutter. In an exemplary embodiment, the subsequent application of the small catch voltage to the left shutter 106 prevents the liquid crystals in the left shutter from rotating too far during the opening of the left shutter 106.

If in 264 if is determined that the left intermittent period has passed, then the process proceeds to 266. The left intermittent period may specify a time period for waiting before the left shutter 106 may be closed again. For example, the left intermittent period may specify that the left shutter controller 116 should activate the left shutter 108 to alternate between open and closed for 15 seconds and then wait for 45 seconds before the left shutter 108 may be activated again.

Alternatively, the right shutter 108 may be controlled by the right shutter controller 118 as discussed above in 256-266. In this case, the right shutter 108 may be activated to close intermittently while the left shutter 106 remains open.

If in 212 it is determined that the therapy sequence is configured to stimulate predominantly blink modulation, the process proceeds to 278. Typically, blinking is a reflex of the user intended to lubricate and protect the user's eyes. The user's pattern of blinking (i.e., rate and duration) may be affected by the user's activity (i.e. reduced blink rate while reading, increased blink rate while performing complex cognitive tasks, etc.). Blinking may also connected to the attention focus of the user, where a brief attention lapse may accompany each blink (so that the actual experience of blinking typically goes un-noticed by the user). The activation of the left shutter 106 or right shutter 108 may be perceived by the eye as a sudden appearance of an object close to the user, which may induce the user to blink thereby affecting the lubrication of the eye as well as the user's perception and attention.

If in 278 it is determined that the left shutter 106 will be closed, then in 280, a high voltage is applied to the left shutter 106 by the left shutter controller 116. In an exemplary embodiment, applying the high voltage to the left shutter 106 closes the left shutter. In an exemplary embodiment, the right shutter 108 is already opened; thus, in 280, the left shutter 106 is closed and the right shutter 108 remains open. If in 282 it is determined that the blink time period has passed, then in 284, no voltage followed by a small catch voltage is applied to the left shutter 106 by the left shutter controller 116. For example, the blink time period for the left shutter 106 may be sufficiently long (e.g., 10 milliseconds) to allow for the left eye of the user to register the left shutter 106 as an object close to the user. In an exemplary embodiment, applying no voltage to the left shutter 106 starts opening the left shutter. In an exemplary embodiment, the subsequent application of the small catch voltage to the left shutter 106 prevents the liquid crystals in the left shutter from rotating too far during the opening of the left shutter 106. As a result, in 232, the left shutter 106 is opened and the right shutter 108 remains open.

Alternatively, the right shutter 108 may be controlled by the right shutter controller 118 as discussed above in 278-286. In this case, the right shutter 108 may be closed for the blink time period and then opened while the left shutter 106 remains open.

In one or more exemplary embodiments, the active glasses 104 may be implemented as described in one or more of the following: U.S. Patent Publication 2010-0177254, U.S. Patent Publication 2010-0157178, U.S. Patent Publication 2010-0157031, U.S. Patent Publication 2010-0157029, U.S. Patent Publication 2010-0157028, U.S. Patent Publication 2010-0149636, U.S. Patent Publication 2010-0157027, U.S. Patent Publication 2010-0149320, U.S. Patent Publication 2010-0165085, U.S. Patent Publication 2010-0245693, and U.S. Patent Publication 2011-0199464, the disclosures of all of which are incorporated herein by reference.

In an exemplary embodiment, a computer readable program product stored on a tangible storage media may be used to facilitate any of the preceding embodiments. For example, embodiments of the invention may be stored on a computer readable medium such as an optical disk (e.g., compact disc, digital versatile disc, etc.), a diskette, a tape, a file, a flash memory card, or any other computer readable storage device. In this example, the execution of the computer readable program product may cause a processor to perform the methods discussed above with respect to FIG. 2A-2F.

In an exemplary embodiment, the system 100 and method 200 of FIGS. 1 and 2A-2F may be to provide therapy for various conditions as described below.

### Epilepsy

In an exemplary embodiment, the system 100 and method 200 may provide treatment for different types of epilepsy.

Light sensitive epilepsy—10% of childhood epilepsy cases are light sensitive. To these patients artificial as well as natural occurring visual stimuli may initiate a seizure. Such seizures may be prevented by occluding one eye of the patient (e.g., stimulating pupil size modulation as discussed above). In this case, long term of the glasses may cause the user to be de-sensitized to flickering lights, eliminating or reducing future seizures.

By using active glasses 104 one eye can be occluded intermittently and at the same time not be visually eliminated. Typically, the duration from initiation of a seizure inducing visual pattern until the development of a seizure is about five seconds. In an exemplary embodiment, activation patterns for treating epilepsy can be either at low frequencies (below about two Hz) or at high frequencies (above about 50 Hz). Examples of low frequency activation patterns may be (1) occlusion of one eye for once second every four seconds or (2) occlusion of alternating eyes, where each eye is occluded for one second in every ten seconds. In these examples, occlusion times may be shorter, for example, up to 0.1 seconds. Examples of high frequency activation patterns may be (1) short occlusions (i.e., approximate five milliseconds) at frequencies that are above the maximal perceived frequency (i.e., approximately fifty Hz), or below the maximal perceived frequency (i.e., approximately five Hz)

Light induced epilepsy—light induced seizures of light sensitive epilepsy (as discussed above) are typically elicited by a flickering at a frequency of about five to 30 Hz. Light stimulation at said frequencies naturally occurs in a wide range of scenarios such as looking out from a driving car, playing video games, etc.

In an exemplary embodiment, by applying a steady flickering using the active glasses 104, stroboscopic effects can be achieved to thereby reduce the perceived frequency of ambient flickering. By reducing the incoming scene to non-seizure priming frequencies, seizures may be prevented. In this case, the activation pattern should include short occlusions at about 50 Hz or higher.

Refractory epilepsy—in the 1990's, intermittent unilateral vagal nerve stimulation therapies were developed that may reduce the occurrence of epilepsy in selected patient populations. The exact mechanism of this therapy has not yet to be defined, although efficacy is well proven.

In an exemplary embodiment, by using the active glasses 104 seizures may be prevented by optical stimulation of the large optical nerve pathway providing a similar effect as electrical stimulation of the vagus nerve (i.e., periodic stimulation to a large cranial nerve). For example, the active glasses 104 may be used to provide constant input that of activity (e.g., flickering that induces inhibitory brain activity) to be inhibited by the brain, which is learned by the brain and applied to inhibit epilepsy. The active glasses 104 allow the brain to be trained without surgery or drugs.

In an exemplary embodiment, activation patterns for refractory epilepsy may be (1) high frequency flickering for 15 seconds every minute; (2) low frequency flickering with short occlusion times (e.g., approximately 0.1 seconds); and (3) low frequency flickering with occlusion times that are as long as 10 seconds. In an exemplary embodiment, the active glasses 104 may be used to prevent seizures, abort seizures and enhance the aura before seizures to allow for patient preparation.

In an exemplary embodiment, the active glasses 104 described above may be used as a diagnostic tool to identify patients that may respond to vagus nerve stimulation (VNS) or other nerve stimulation therapy. In this case, a patient that suffers from refractory epilepsy and is considering implantation of a VNS system may initially use the active glasses 104 for a preliminary period of days or weeks (e.g., 21 days). The effect of the active glasses 104 therapy on the frequency of epileptic attacks and/or on the visually evoked potentials or other signs and symptoms may be assessed, and the implantation of a VNS system may then be considered based on the results. Typically, a patient that fails to show any response to active glasses 104 therapy is expected to be refractive to VNS therapy, indicating that the patient may want to consider forgoing the implantation procedure.

### Depression

It has been shown that prolonged illumination (e.g., a few hours) each morning may alleviate symptoms of depression. In an exemplary embodiment, the active glasses 104 are configured to provide variances in illumination (as opposed to fixed strong illumination typically used in light treatment for depression) to alleviate depression.

In an exemplary embodiment, by using the active glasses 104, it is possible to cause visible changes in the perceived illumination to an individual eye or to both eyes without external illumination (i.e., additional illumination that is not ambient). In this case, therapy may be more effective and less cumbersome than using the typical light therapy schemes that use external illumination. For example, an activation pattern for depression may be increasing the transparency of the glasses for an extended time period (e.g., up to three hours), followed by a decrease in transparency for a similar time period. In this example, the duty cycle of the active glasses 104 may be short as five minutes or as long as 24 hours. Further, the change in transparency should be subtle to allow for the active glasses 104 to be used indoors at both levels of illumination.

As discussed above with respect to epilepsy, vagal nerve stimulation may be used for the treatment of depression. Specifically, it is postulated that the unilateral intermittent stimulation of an essential pathway in the nervous system may stimulate the nervous system, resulting in higher levels of alertness, serotonin and mood. Using the active glasses 104 technique it may be possible to stimulate the visual pathway to obtain these results while causing minimal discomfort for the patient (i.e., without surgery). In addition to the anti-depressive activity, this therapy is believed to also be efficient in the treatment of bi-polar disorders (e.g., true bi-polar disorder, mood swings, etc.).

### Age Related Macular Degeneration (AMD) and Other Retinopathies

Patients with AMD have a shortage in active neurons in their visual center. Current therapeutic schemes focus on (1) activating the peripheral vision by re-directing the incoming visual image or (2) direct electrical activation of the macular neurons by retinal implants.

In an exemplary embodiment, by using the system 100, maximized activation of the remaining macular photoreceptors may be achieved using ambient light and the active glasses 104. In this case, although the underlying cause of AMD is not reversed, the patient may experience relief from the symptoms of AMD (e.g., loss of vision) is by enhancing the activity of the remaining cells and enhancing the user's sight. In an exemplary embodiment, stimulation of the optic nerve as described above may increase the activity of the visual system thereby enhancing perception of the image and light (e.g., by attenuating lateral inhibition).

In an exemplary embodiment, each eye is occluded separately for periods of about 0.2 to two seconds, which followed by a similar time period with the lens in a transparent state. In some embodiments, when one eye has significantly superior sight than the other eye, the periods of occlusion may be different for each eye (e.g., the stronger eye receives shorter occlusions than the weaker eye).

In some embodiment, the transition of the shutters 106 and 108 from transparent to opaque is relative (e.g., some opacity remaining in the transparent state and some transparency remaining also in the opaque state). For example, the contrast between the modes may vary depending on the treatment (e.g., the contrast may be at least 100 such as 700 or considerably lower such as 10). In an exemplary embodiment, the precise control of transparency possible with active glasses 104 may be therapeutic for an AMD patient. In these patients too much or too little illumination may further reduce visual performance and cause a considerable degree of discomfort. By allowing the patient to control overall transparency using the active glasses 104, the optimal illumination level for subjective visual performance may be achieved.

### Central Nervous System (CNS) Hyper Excitation

Optic stimulation as described above may be used to stimulate the corresponding visual and neural pathways, which may elicit inhibitory effects on the brain. Specifically, generally inhibition of higher centers of the brain may be elicited while lower centers are stimulated. The optic stimulation causes modulation of the brain's response to input, where the modulation may have a beneficial effect for attention deficit hyperactivity disorder (ADHD) patients. Further, modulating the excitatory patterns of the brain may also be beneficial to relive conditions such as chronic pain, eating disorders, migraine, mania, aggressiveness, and obsessive compulsive disorders.

In exemplary embodiment, by providing a repetitive useless incoming signal over a period of hours using the active glasses 104, higher centers of the brain are forced to practice increased inhibition to maintain normal activity. In this case, undesired hyper excitation may be attenuated (e.g., attenuation of alertness to promote sleep, attenuation of chronic pain, attenuation of response to changes in blood flow to prevent migraine attacks, attenuation of anxiety, attenuation of ADHD symptoms, and attenuation of obsessive thoughts and behaviors).

In some embodiments, the active glasses 104 may be utilized before the inhibitory result is desired. For example, the active glasses 104 may be utilized for at least 30 minutes before sleep is desired. In another example, the active glasses 104 may be utilized at least 30 minutes before and during the period where learning and concentration are desired (e.g., school day, work task, etc.).

In some embodiments, the glasses are activated at cycles that correlate to the frequency of the “default network” frequency—0.01 Hz to 0.1 Hz, i.e. activated briefly (for 2 seconds) once every 10 to 100 seconds. In these embodiments, the continued activation at the default frequency helps the brain to filter the activity of the default network, promoting the ability for focused attention.

In other embodiments, the therapeutic parameters described for sleeping disorders are applied to persons suffering from ADHD or ADD, normalizing sleeping patterns and brain activity cycles, to treat the condition.

### Strabismus

Strabismus is a vision problem characterized by a misalignment of the eyes (i.e., the eyes do not look at the same point at the same time). Proper alignment of both eyes may be desired for depth perception and cosmetic reasons.

In an exemplary embodiment, by using the active glasses 104 to intermittently occlude the eye that is on target (e.g., by stimulating pupil size modulation) the deviating eye may be encouraged to acquire the target of the user's gaze in order to maintain visual continuity. After extended use of the active glasses 104, the user learns to maintain both eyes on a target in order to avoid frequent acquisitions of the deviating eye (and the stutter in vision that accompanies such a rapid acquisition).

In some embodiments, the active glasses 104 intermittently occlude one eye. In other embodiments, the active glasses 104 alternatively occludes both eyes (i.e., one shutter is closed while the other is open). In either case, the time that both eyes are free to obtain the full visual image may be at least equal to the time a single eye is occluded. In an exemplary embodiment, the active glasses 104 occlude the weaker eye for approximately 20 seconds each minute, which may significantly improve visual acuity and depth perception of the weaker eye. Further, with prolonged use of the active glasses 104, improvements in strabismus may also be observed in the user.

### Cortical Rehabilitation

Optic nerve stimulation may be used to assist in rehabilitating a severed visual cortex, such as in cortical blindness following ischemic brain damage. Rehabilitation may be facilitated by performing short stimulations with white light and/or patterns in an attempt to regain function of severed brain areas. Current procedures perform stimulations under special conditions, where the patient is restrained in a dark room with his eyes and head fixed. Due to the hardship involved, typical stimulation therapy typically includes a daily treatment of no more than two hours.

In an exemplary embodiment, the active glasses 104 may be used to induce optic stimulation for longer periods of time (e.g., as much as all waking hours) while causing minimal disturbance to the patient. In some embodiments, the activation pattern includes about 100 to 150 milliseconds of light (e.g., ambient light) followed by approximately 0.5 to five seconds of occlusion. This activation pattern may be applied to each eye separately (to allow for normal eye function and increased user compliance) while the other eye is (1) at constant rest or (2) at an activation level similar to the one described above for AMD therapy.

In some embodiments, intensive therapy courses may be applied where both eyes receive similar activation patterns simultaneously. For example, the intensive therapy courses may be applied for a few hours a day either in one session or in multiple short sessions occurring occasionally throughout the day.

### Reading Disorders

The normal development of reading skills is a complex and multistage process. While acquiring reading skills demands a certain skill and structure set, the act of fluently reading involves other (higher) functions. When two visual inputs are presented near in time, one of the visual inputs may be ignored by the brain, which is a phenomenon described as attentional blink. Unrelated visual motions and flicker may attenuate the attentional blink thereby alleviating various reading disorders including dyslexia and reading difficulties usually associated with ADHD. In an exemplary embodiment, the active glasses 104 apply additional disturbing flicker over one or both eyes to improve reading capabilities.

In some embodiments, the disturbing flickers are applied frequently (i.e., fast enough to affect the attention blink between letters or words while reading). For example, the active glasses 104 may applying to each eye a brief flicker of ten milliseconds for every 40 millisecond time period. In this case, the flickering is synchronized between the eyes so that at any point in time (1) at least one eye is open and (2) the timing between the right eye and left eye occlusions is approximately equal through the cycle.

### Optic Disturbances (Halos and Glares)

Various visual disturbances are influenced by the size of the pupil. As in photography, the diameter of the opening in the iris determines the aperture of the camera (or eye in our case) and has significant effect on the resulting optic image (e.g., depth of field, focus, etc.). The aperture may be even more influential in cases where optic aberrations are present, such as in patients after Lasik surgery, patients with artificial intra-ocular lenses, patients using multi focal optics, and patients suffering from cataracts.

While a smaller aperture generally results in sharper optic images, the amount of incoming light is reduced and, thus, the image quality may be reduced. As opposed to cameras, humans are typically incapable of controlling their pupil dilation to improve their vision.

In an exemplary embodiment, the active glasses 104 are used to reduce the size of the pupils as discussed above in order to improve vision in cases where optic aberrations are expected (e.g., stimulating pupil size modulation). In some embodiments, a small light source may be intermittently applied to create the constriction. In other embodiments, the active glasses 104 apply intermittent occlusions over the eye to expose the papillary reflex arc to variations in light intensity, resulting in a pupil diameter/light intensity ratio that is larger than the ratio obtained without the flickering. For example, the active glasses 104 may be configured to synchronize alternating occlusions (i.e., one shutter closed while the other shutter is open) with a duration of 250 milliseconds at a rate of 0.1 Hz for each eye.

### Sleeping Disorders

The frequency of brain waves may be modulated by applying visual and audio inputs at desired frequencies. While some frequencies are known to correlate with sleep (i.e., 0.5 Hz to 4 Hz, known as Delta waves), other waves correlate to alertness (i.e., 13 Hz to 30 Hz, known as Beta waves), and yet other waves correlate to relaxation (8 Hz to 13 Hz, known as Alpha waves).

In an exemplary embodiment, the active glasses 104 are used to provide visual stimulation at frequencies correlating to the desired effect for long durations, while the user is allowed to function normally. Examples of uses include alleviating insomnia, preventing sleepiness while driving, etc., increasing concentration and aiding in learning, relaxing the patient (i.e., reducing anxiety). In an exemplary embodiment, the active glasses 104 are configured to be occluded such that the overall occlusion time is less than 30% while the frequency conveyed by the active glasses 104 is set to achieve the desired effect (e.g., alertness, relaxation, etc.). For example, the active glasses 104 may provide 300 milliseconds of occlusion for every second to help induce and maintain sleep.

In some embodiments, the glasses 104 are utilized at specific timings within the circadian cycle, to entrain the circadian cycle and normalize sleeping patterns, i.e., the glasses are routinely used at sleep inducing parameters, for example each night two hours prior to the desired bed time even if the actual sleeping time is much delayed. The routine use of the glasses 104 may result in an eventual shift of the actual sleeping time towards the desired sleeping time. In some embodiments, the glasses 104 are activated in the same parameters in cycles that are roughly the length of sleeping cycle (e.g., 80 to 120 minutes). For example, the glasses 104 may be activated for 10 minutes, followed by 80 minutes of rest, in cycles throughout the day to induce, enforce, and maintain normal brain activity cycles necessary for normal sleep.

In all of the aforementioned treatments, occlusions may be applied to either eye, to both eyes, or to specific regions of each retina. In some embodiments, the active glasses 104 may be used in conjunction with optical maneuvers (e.g., laser projection) to achieve high resolution retinal occlusion or stimulation. Further, occlusion may be achieved by either occlusion of the eye or by occlusion (or flickering) of a light source such as ambient light or the light generated by a computer screen or television.

In some embodiments, the system 100 may achieve a differential effect for each eye (or area within the same eye) by combining flickering of a light source with one or more of the previously described occlusions, where the timing for each eye or area of the eye is different. In other embodiments, intermittently polarizing ambient light combined with polarizing glasses (distinct for each eye or area of the eye) may be used to achieve a similar effect.

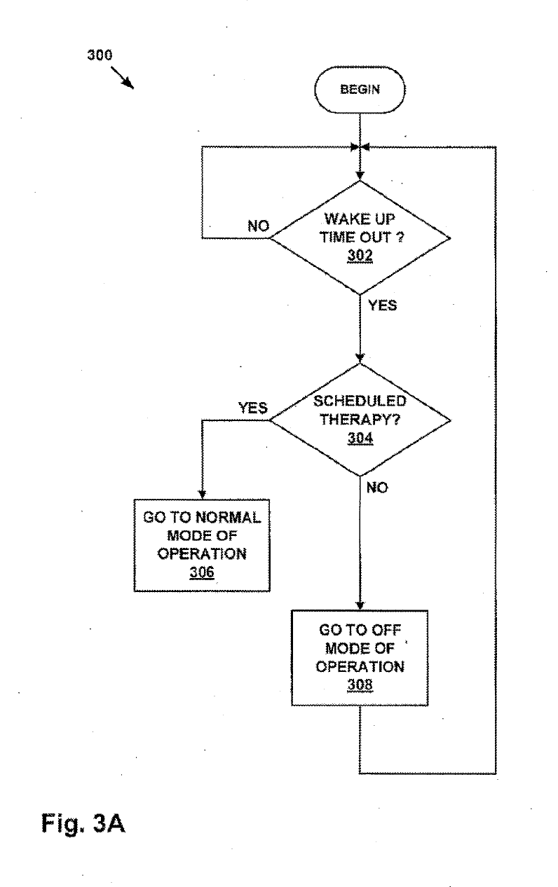

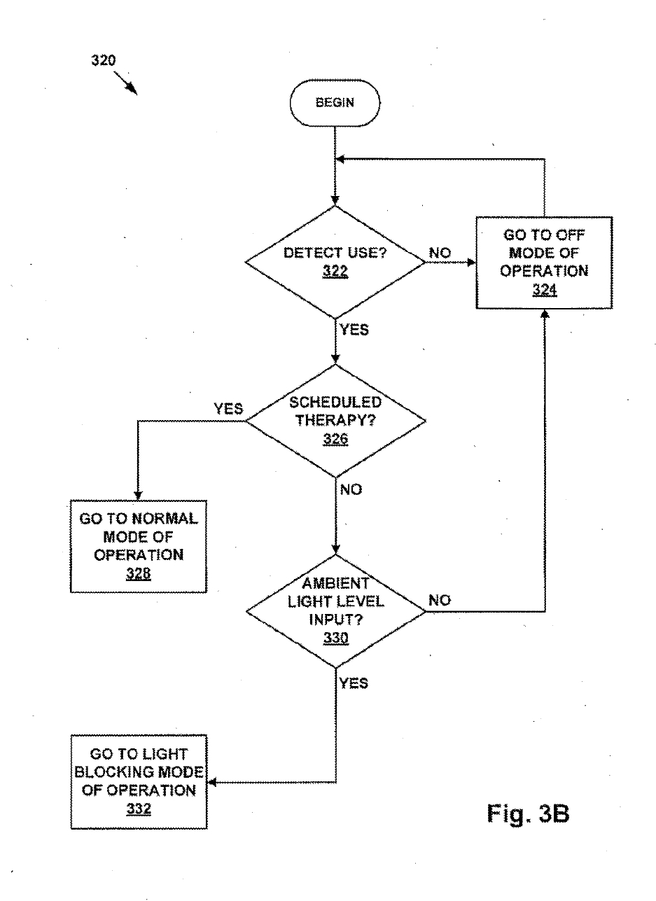

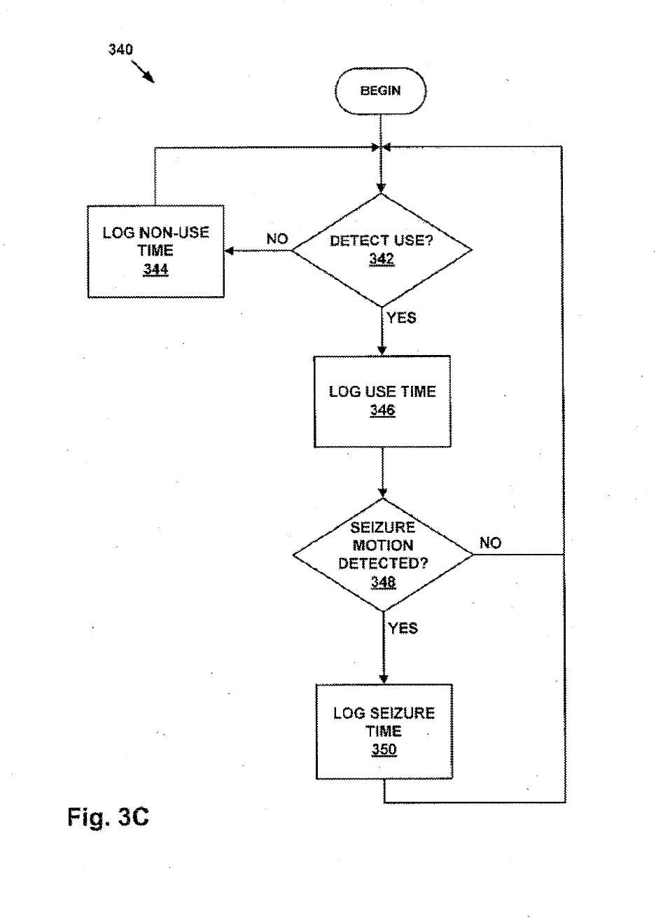

Referring now to FIG. 3A, in an exemplary embodiment, during the operation of the system 100, the active glasses 104 implement a method 300 of operation in which, in 302, the CPU 114 of the active glasses 104 checks for a wake up mode time out. In an exemplary embodiment, the presence of a wake up mode time out in 302 is provided by a predetermined time period.

If the CPU 114 detects a wake up time out in 302, then the CPU checks for the presence or absence of a scheduled therapy in 304. If the CPU 114 detects that a therapy is scheduled in 304, then the CPU places the active glasses 104 in a NORMAL MODE of operation in 306. In an exemplary embodiment, in the NORMAL MODE of operation, the active glasses implement, at least portions of, the method 200, obtaining a therapy sequence and stimulating optic nerves using one or more visual properties.

If the CPU 114 does not detect a scheduled therapy in 304, then the CPU places the active glasses 104 in an OFF MODE of operation in 308 and then, in 302, the CPU checks for a wake up mode time out. In an exemplary embodiment, in the OFF MODE of operation, the active glasses do not provide the features of NORMAL or CLEAR mode of operations.

In an exemplary embodiment, the method 300 is implemented by the active glasses 104 when the active glasses suspend operation after a predetermined time period of inactivity.

Referring now to FIG. 3B, in an exemplary embodiment, during the operation of the system 100, the active glasses 104 implement a method 320 of operation in which, in 322, the CPU 114 of the active glasses 104 checks for use of the active glasses 104 by a user. In an exemplary embodiment, the detection of use in 322 is determined using a motion sensor such as an accelerometer, a gyroscope, a proximity sensor, etc. For example, the active glasses 104 may be detected as being in use when a proximity sensor of the active glasses 104 activates as they are placed on the face of the user. In this example, the active glasses 104 may be detected as being in non-use when the proximity sensor of the active glasses 104 is deactivated for a predetermined period of time.

If the CPU 114 does not detect a use in 322, then the CPU places the active glasses 104 in an OFF MODE of operation in 324 and then, in 322, the CPU checks for use of the active glasses 104 by the user. In an exemplary embodiment, in the OFF MODE of operation, the active glasses 104 do not provide the features of NORMAL or CLEAR mode of operations.

If the CPU 114 detects a use in 322, then the CPU checks for the presence or absence of a scheduled therapy in 326. If the CPU 114 detects that a therapy is scheduled in 326, then the CPU places the active glasses 104 in a NORMAL MODE of operation in 328. In an exemplary embodiment, in the NORMAL MODE of operation, the active glasses 104 implement, at least portions of, the method 200, obtaining a therapy sequence and stimulating optic nerves using one or more visual properties.

If the CPU 114 does not detect a scheduled therapy in 326, then the CPU checks for the presence or absence of ambient light level input in 330. If the CPU 114 detects ambient light level input in 330, then the CPU places the active glasses 104 in a LIGHT BLOCKING MODE of operation in 332. In an exemplary embodiment, in the LIGHT BLOCKING MODE of operation, the active glasses 104 implement a shutter sequence for preventing ambient light from damaging or discomforting the eyes of the user.

In some embodiments, the ambient light level input may be ambient light levels detected by a light sensor of the active glasses 104. In this case, the shutter sequence of the active glasses 104 may be automatically adapted to varying ambient light levels detected by the light sensor. In other embodiments, the ambient light level input may be user input to either darken or lighten the shutters of the active glasses 104 to alter the amount of ambient light reaching the eyes of the user. In either case, the amount of ambient light blocked by the active glasses 104 may be controlled by increasing or decreasing a frequency that the shutters switch between an open and closed state.

If the CPU 114 does not detect an ambient light level input in 330, then the CPU places the active glasses 104 in an OFF MODE of operation in 324 and then, in 322, the CPU checks for use of the active glasses 104 by the user.

In an exemplary embodiment, the method 320 is implemented by the active glasses 104 when the active glasses suspend operation after a predetermined time period of non-use.

Referring now to FIG. 3C, in an exemplary embodiment, during the operation of the system 100, the active glasses 104 implement a method 340 of operation in which, in 342, the CPU 114 of the active glasses 104 checks for use of the active glasses 104 by a user. In an exemplary embodiment, the detection of use in 342 is determined using a motion sensor such as an accelerometer, a gyroscope, etc.

If the CPU 114 does not detect a use in 342, then the CPU 114 logs the non-use time in 344 and then, in 342, the CPU checks for use of the active glasses 104 by the user. In an exemplary embodiment, the non-use time is logged in the memory 115 of the active glasses 104.

If the CPU 114 detects a use in 342, then the CPU 114 logs the use time in 346. In an exemplary embodiment, the use time is logged in the memory 115 of the active glasses 104. In 348, the CPU 114 of the active glasses 104 checks for a seizure motion of the active glasses 104 by a user. In an exemplary embodiment, the detection of a seizure motion in 348 is determined using a motion sensor such as an accelerometer, a gyroscope, etc. A seizure motion may correspond to irregular thrashing or convulsions of the user detected by the motion sensor.

If the CPU 114 detects a seizure motion in 348, then the CPU 114 logs the seizure motion in 350. In an exemplary embodiment, the seizure motion is logged in the memory 115 of the active glasses 104. If the CPU 114 does not detect a seizure motion in 348, then the CPU 114 checks for use of the active glasses 104 by the user in 342.

In an exemplary embodiment, the method 340 is implemented by the active glasses 104 in conjunction with one or more of the methods (e.g., 200 of FIG. 2, 300 of FIG. 3A, 320 of FIG. 3B) described above in order to improve the functionality of the active glasses 104. Specifically, use and non-use times may be logged and then used to customize therapy sequences provided as discussed above with respect to FIG. 2. For example, the duration of the therapy sequences may be adjusted to accommodate for periods of non-use (e.g., extending a therapy sequence to compensate for a missed session, shortening a therapy sequence so that the user may reacclimate after an extended period of non-use, etc.)

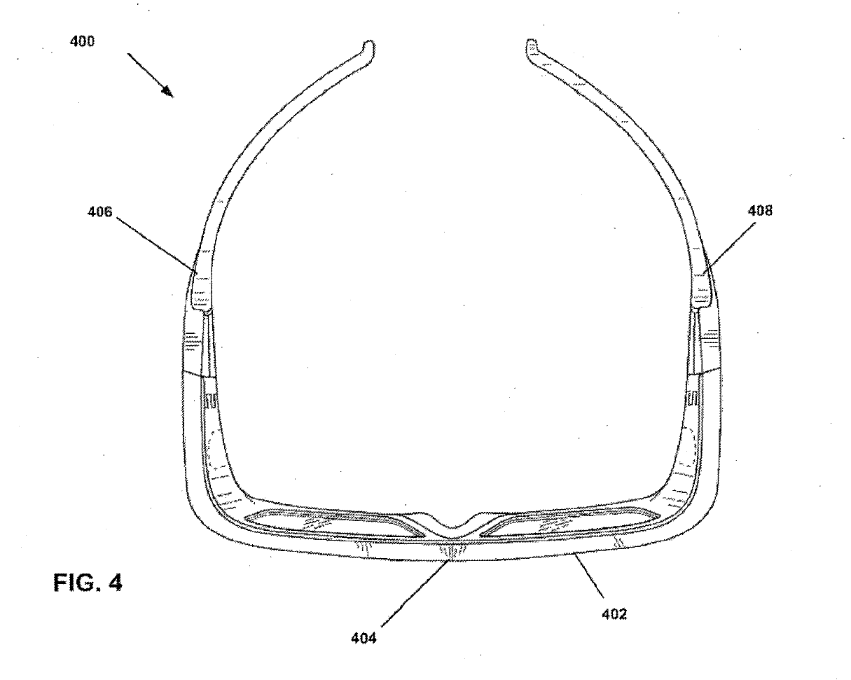

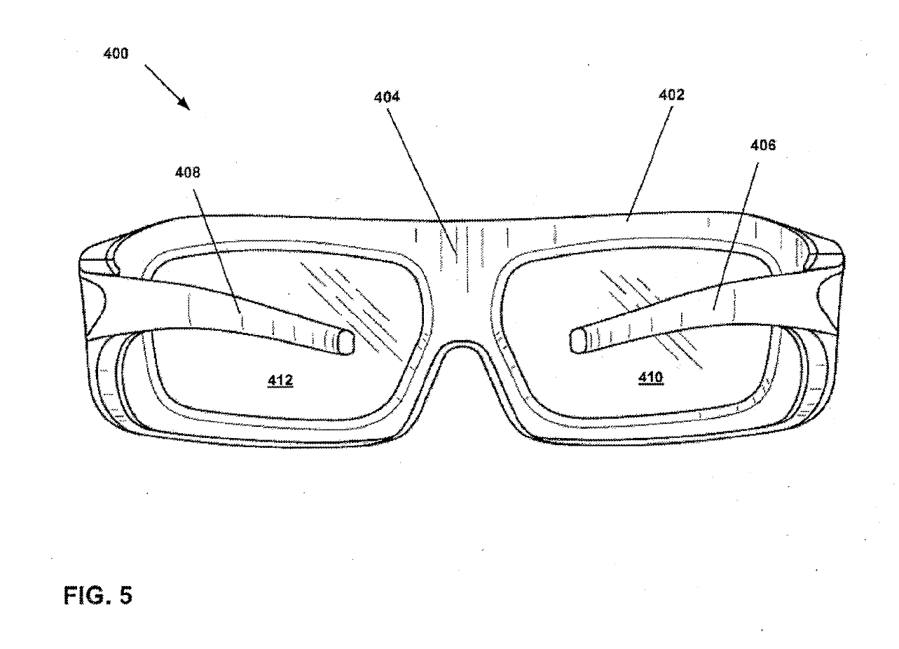

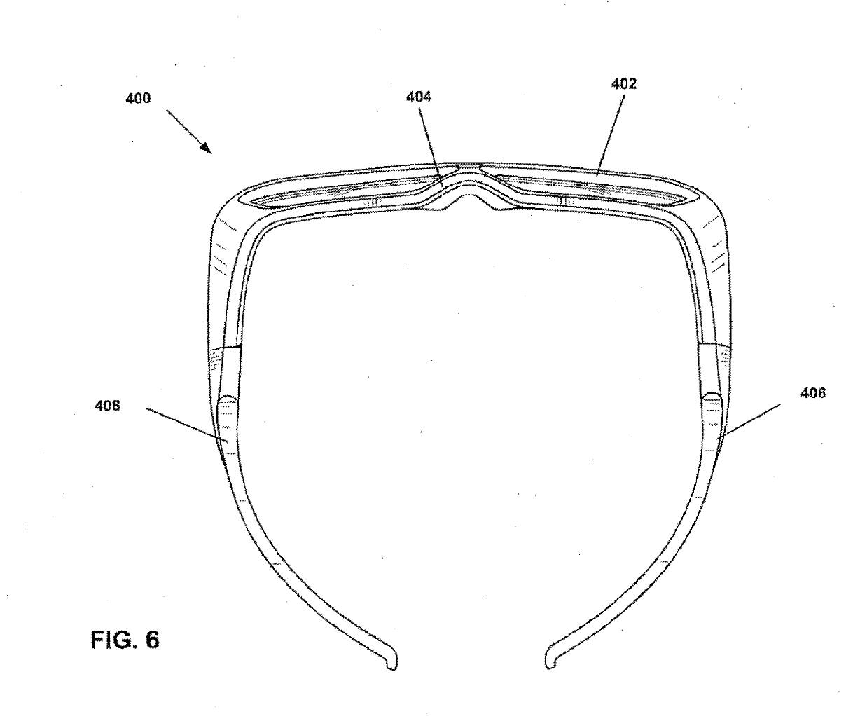

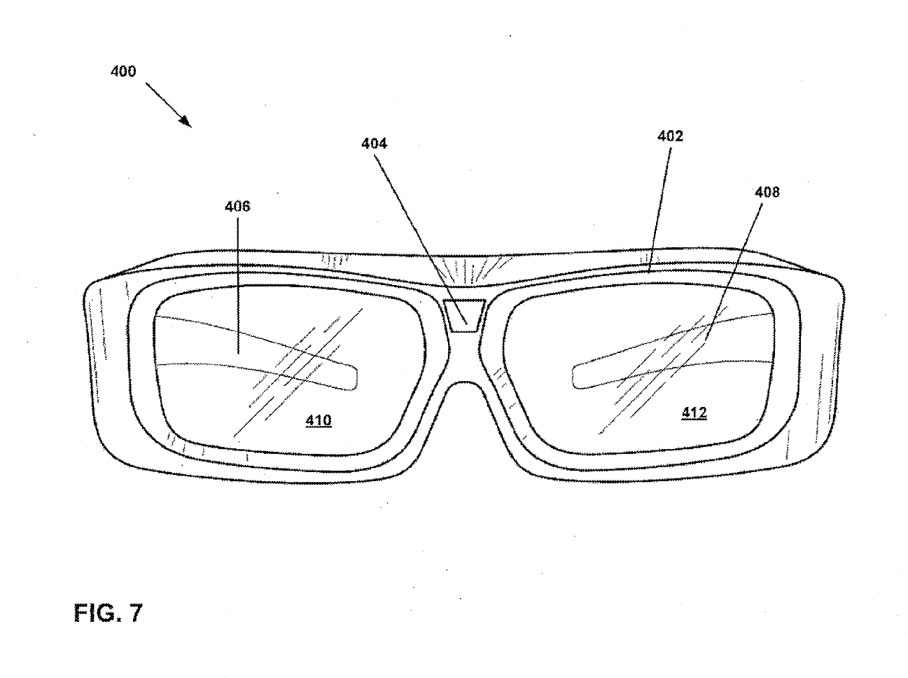

Referring now to FIGS. 4-9, in an exemplary embodiment, one or more of the active glasses 104 and 400 include a frame front 402, a bridge 404, right temple 406, and a left temple 408. In an exemplary embodiment, the frame front 402 houses the control circuitry and power supply for one or more of the active glasses 104 and 400, as described above, and further defines right and left lens openings, 410 and 412, for holding the right and left liquid crystal shutters described above. In some embodiments, the frame front 402 wraps around to form a right wing 402a and a left wing 402b. In some embodiments, at least part of the control circuitry for the active glasses 104 and 400 are housed in either or both wings 402a and 402b.

In an exemplary embodiment, the right and left temples, 406 and 408, extend from the frame front 402 and each have a curved shape, with the far ends of the temples being spaced closer together than at their respective connections to the frame front. In this manner, when a user wears the active glasses 104 and 400, the ends of the temples, 406 and 408, hug and are held in place on the user's head.

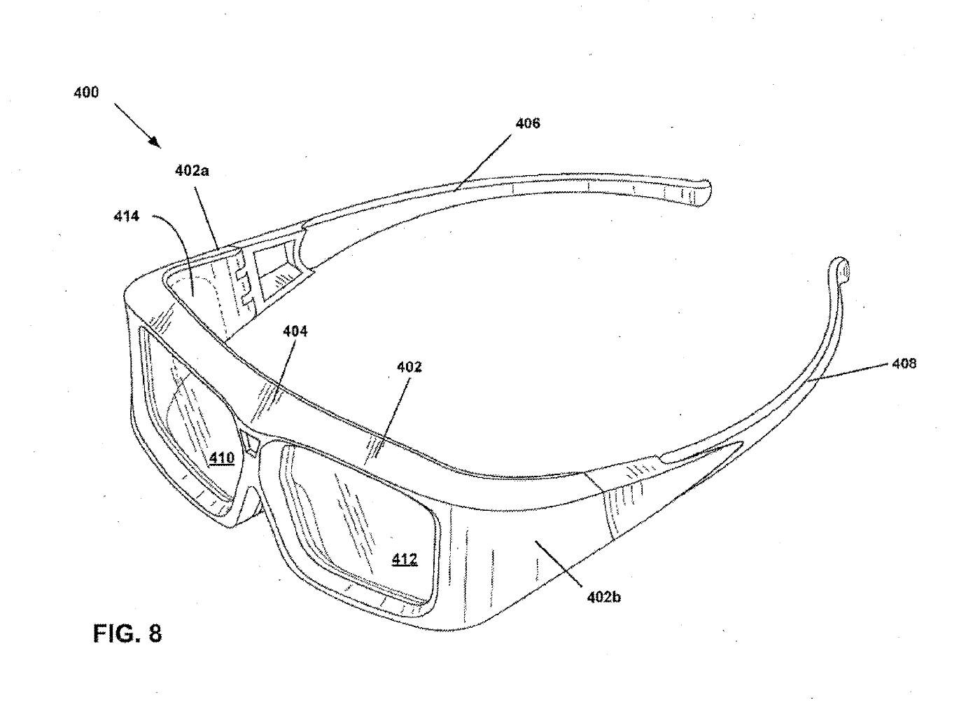

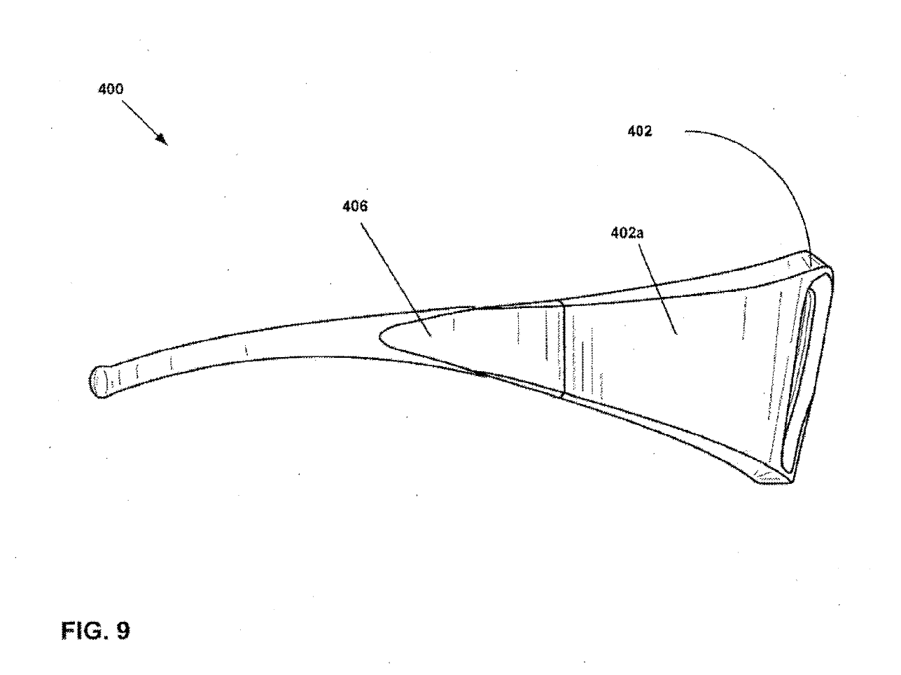

Referring now to FIGS. 8-9, in an exemplary embodiment, the control circuitry for one or more of the active glasses 104 and 400 is housed in the frame front, which includes the right wing 402a, and the battery is housed in the right wing 402a. Furthermore, in an exemplary embodiment, access to the battery 120 of the active glasses 400 is provided through an opening, on the interior side of the right wing 402a, that is sealed off by a cover 414 that may include a seal (not shown) for mating with and sealingly engaging the right wing 402a.

Referring to FIG. 9, in some embodiments, the battery is located within a battery cover assembly formed by cover 414 and cover interior (not shown). Battery cover 414 may be attached to battery cover interior by, for example, mechanical means. Contacts (not shown) may stick out from the batter cover interior to conduct electricity from the battery 120 to contacts located, for example, inside the right wing 402a. The control circuitry and battery of the active glasses 104 and 400 may be sealed off from the environment by the engagement of the cover 414 with the right wing 402a of the active glasses 400.

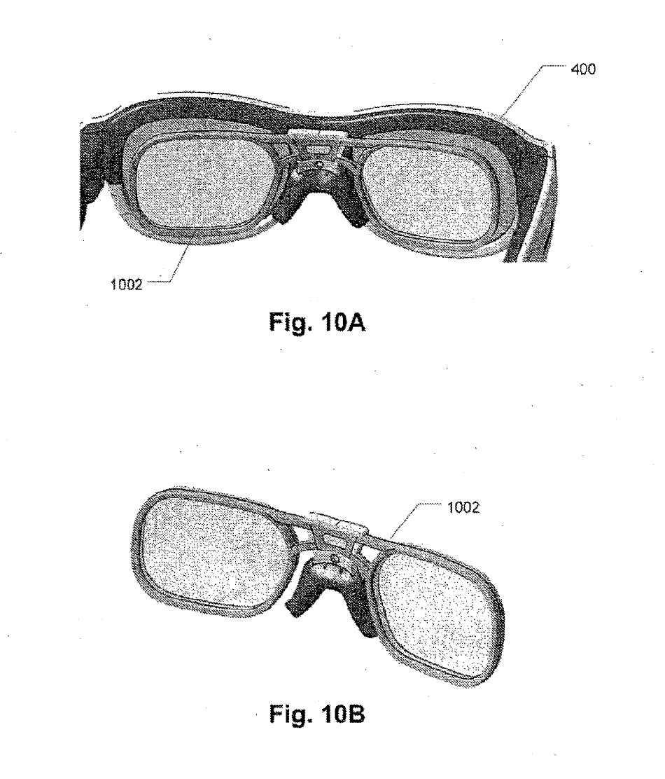

Referring now to FIGS. 10A-10B, in an exemplary embodiment, the active glasses 400 are attached to a prescription attachment 1002. In an exemplary embodiment, the prescription attachment 1002 includes corrective lenses for use with the active glasses 400. The corrective lenses are positioned behind the shutters of the active glasses 400 such that the vision of the user is corrected while using the active glasses 400.

A liquid crystal shutter has a liquid crystal that rotates by applying an electrical voltage to the liquid crystal and then the liquid crystal achieves a light transmission rate of at least twenty-five percent in less than one millisecond. When the liquid crystal rotates to a point having maximum light transmission, a device stops the rotation of the liquid crystal at the point of maximum light transmission and then holds the liquid crystal at the point of maximum light transmission for a period of time. A computer program installed on a machine readable medium may be used to facilitate any of these embodiments.

It is understood that variations may be made in the above without departing from the scope of the invention. While specific embodiments have been shown and described, modifications can be made by one skilled in the art without departing from the spirit or teaching of this invention. The embodiments as described are exemplary only and are not limiting. Many variations and modifications are possible and are within the scope of the invention. Furthermore, one or more elements of the exemplary embodiments may be omitted, combined with, or substituted for, in whole or in part, one or more elements of one or more of the other exemplary embodiments. Accordingly, the scope of protection is not limited to the embodiments described, but is only limited by the claims that follow, the scope of which shall include all equivalents of the subject matter of the claims.

1. A system for stimulating optic nerves, the system comprising:
a left shutter controller operably coupled to a left liquid crystal viewing shutter for controlling the left liquid crystal viewing shutter;
a right shutter controller operably coupled to a right liquid crystal viewing shutter for controlling the right liquid crystal viewing shutter;
a charge pump operably coupled to the left and right shutter controllers for amplifying an output voltage of a battery power supply to provide control input signals to each of the left and right shutter controllers;
a central processing unit (CPU) operably coupled to the left and right shutter controllers for controlling an operation of the left and right shutter controllers, the CPU adapted to:
obtain a therapy sequence utilizing one or more properties of a visual system to stimulate the optic nerves of a user; and
control a shutter activity of the left and right shutter controllers based on the therapy sequence.

2. The system of claim 1, wherein the one or more properties of the visual system are selected from a group consisting of enhanced contrast, darkness adaptation, neighbor cell inhibition, pupil size modulation, and blink modulation.

3. The system of claim 1, wherein the one or more properties of the visual system includes enhanced contrast, the enhanced contrast of the user being utilized by alternating each of the left and right crystal viewing shutters between an opaque state and a transparent state.

4. The system of claim 3, wherein the left and right crystal viewing shutters are in the transparent state for no longer than 0.1 seconds.

5. The system of claim 1, wherein the one or more properties of the visual system includes darkness adaptation, the darkness adaptation of the user being utilized by controlling one of the left and right crystal viewing stutters to be in an opaque state for at least 30 seconds.

6. The system of claim 1, wherein the one or more properties of the visual system includes neighbor cell inhibition, the neighbor cell inhibition of the user being decreased by controlling each of the left and right crystal viewing stutters to be in an opaque state for at least 20 seconds for every two minute time period.

7. The system of claim 1, wherein the one or more properties of the visual system includes pupil size modulation, the pupil size modulation of the user being utilized by intermittently alternating each of the left and right crystal viewing shutters between an opaque state and a transparent state.

8. The system of claim 1, wherein the one or more properties of the visual system includes blink modulation, the blink modulation of the user being utilized by controlling at least one of the left and right crystal viewing stutters to be in an opaque state for a predetermined time that allows the user to perceive the opaque state as a nearby object.

9. The system of claim 1, wherein the therapy sequence is adapted for a treatment of epilepsy, and wherein the one or more properties of the visual system are utilized by cycling the left crystal viewing shutter between an opaque state of less than about 0.1 seconds and a transparent state at a first frequency of less than about two hertz, the left crystal viewing shutter being in the opaque state for about one second of a four second time period.

10. The system of claim 1, wherein the therapy sequence is adapted for a treatment of epilepsy, and wherein the one or more properties of the visual system are utilized by alternately:
cycling the left crystal viewing shutter between an opaque state of less than about 0.1 seconds and a transparent state at a first frequency of less than about two hertz, the left crystal viewing shutter being in the opaque state for about one second of a ten second time period; and
cycling the right crystal viewing shutter between the opaque state of less than about 0.1 seconds and the transparent state at a second frequency of less than about two hertz, the right crystal viewing shutter being in the opaque state for about one second of the ten second time period.

11. The system of claim 1, wherein the therapy sequence is adapted for a treatment of epilepsy, and wherein the one or more properties of the visual system are utilized by alternately:
cycling the left crystal viewing shutter between an opaque state of about five milliseconds and a transparent state at a first frequency of greater than about 50 hertz; and
cycling the right crystal viewing shutter between the opaque state of about five milliseconds and the transparent state at a second frequency of greater than about 50 hertz.

12. The system of claim 1, wherein the therapy sequence is adapted for a treatment of depression, and wherein the one or more properties of the visual system are utilized by alternately:
increasing transparency of the left and right crystal viewing shutters for a period of time; and
decreasing the transparency of the left and right crystal viewing shutters for the period of time.

13. The system of claim 1, wherein the therapy sequence is adapted for a treatment of reduced vision, and wherein the one or more properties of the visual system are utilized by alternately:
controlling the left crystal viewing shutter to be in an opaque state and the right crystal viewing shutter to be in a transparent state for a first time range of about 0.2 to 2 seconds; and
controlling the right crystal viewing shutter to be in the opaque state and the right crystal viewing shutter to be in the transparent state for a second time range of about 0.2 to 2 seconds.

14. The system of claim 1, wherein the therapy sequence is adapted for a treatment of strabismus by cycling one of the left and right crystal viewing shutters between an opaque state and a transparent state, the one of the left and right crystal viewing shutters being in the opaque state for about 20 seconds of a one minute time period.

15. The system of claim 1, wherein the therapy sequence is adapted for a treatment of attention deficit hyperactivity disorder by providing a repetitive signal using the left and right crystal viewing shutters over an extended period of time of at least one hour.

16. The system of claim 1, wherein the left liquid crystal viewing shutter and the right liquid crystal viewing shutter have a different polarization orientation.

17. The system of claim 1, wherein the shutter activity modifies light received from an ambient light source.

18. The system of claim 1, wherein the shutter activity modifies light received from an external light source.

19. The system of claim 1, the CPU is further adapted to:
log use times of the system by the user; and
adjust a duration of the therapy sequence based on the logged use times.

20. Active glasses for stimulating optic nerves, comprising:
a left shutter controller operably coupled to a plurality of left liquid crystal viewing (LCV) shutters for controlling the plurality of left LCV shutters, each of the plurality of left LCV shutters positioned in a corresponding region of a left lens of the active glasses;
a right shutter controller operably coupled to a plurality of right LCV shutters for controlling the plurality of right LCV shutters, each of the plurality of right LCV shutters positioned in a corresponding region of a right lens of the active glasses;
a charge pump operably coupled to the left and right shutter controllers for amplifying an output voltage of a battery power supply to provide control input signals to each of the left and right shutter controllers;
a central processing unit (CPU) operably coupled to the left and right shutter controllers for controlling an operation of the left and right shutter controllers, the CPU adapted to:
obtain a therapy sequence utilizing one or more properties of a visual system to stimulate the optic nerves of a user; and
control a shutter activity of the left and right shutter controllers based on the therapy sequence.

21. A method for stimulating optic nerves, the method comprising:
obtaining a therapy sequence utilizing one or more properties of a visual system to stimulate the optic nerves of a user; and
performing a shutter activity based on the therapy sequence by:
controlling a left liquid crystal viewing shutter of active glasses; and
controlling a right liquid crystal viewing shutter of the active glasses.

22. The method of claim 21, wherein the one or more properties of the visual system are selected from a group consisting of enhanced contrast, darkness adaptation, neighbor cell inhibition, pupil size modulation, and blink modulation.

23. The method of claim 21, wherein the one or more properties of the visual system includes enhanced contrast, the enhanced contrast of the user being utilized by alternating each of the left and right crystal viewing shutters between an opaque state and a transparent state.

24. The method of claim 23, wherein the left and right crystal viewing shutters are in the transparent state for no longer than 0.1 seconds.

25. The method of claim 20, wherein the one or more properties of the visual system includes darkness adaptation, the darkness adaptation of the user being utilized by controlling one of the left and right crystal viewing stutters to be in an opaque state for at least 30 seconds.

26. The method of claim 21, wherein the one or more properties of the visual system includes neighbor cell inhibition, the neighbor cell inhibition of the user being decreased by controlling each of the left and right crystal viewing stutters to be in an opaque state for at least 20 seconds for every two minute time period.

27. The method of claim 21, wherein the one or more properties of the visual system includes pupil size modulation, the pupil size modulation of the user being utilized by intermittently alternating each of the left and right crystal viewing shutters between an opaque state and a transparent state.

28. The method of claim 21, wherein the one or more properties of the visual system includes blink modulation, the blink modulation of the user being utilized by controlling at least one of the left and right crystal viewing stutters to be in an opaque state for a predetermined time that allows the user to perceive the opaque state as a nearby object.

29. The method of claim 21, wherein the therapy sequence is adapted for a treatment of epilepsy, and wherein the one or more properties of the visual system are utilized by cycling the left crystal viewing shutter between an opaque state of less than about 0.1 seconds and a transparent state at a first frequency of less than about two hertz, the left crystal viewing shutter being in the opaque state for about one second of a four second time period.

30. The method of claim 21, wherein the therapy sequence is adapted for a treatment of epilepsy, and wherein the one or more properties of the visual system are utilized by alternately:
cycling the left crystal viewing shutter between an opaque state of less than about 0.1 seconds and a transparent state at a first frequency of less than about two hertz, the left crystal viewing shutter being in the opaque state for about one second of a ten second time period; and
cycling the right crystal viewing shutter between the opaque state of less than about 0.1 seconds and the transparent state at a second frequency of less than about two hertz, the right crystal viewing shutter being in the opaque state for about one second of the ten second time period.

31. The method of claim 21, wherein the therapy sequence is adapted for a treatment of epilepsy, and wherein the one or more properties of the visual system are utilized by alternately:
cycling the left crystal viewing shutter between an opaque state of about five milliseconds and a transparent state at a first frequency of greater than about 50 hertz; and
cycling the right crystal viewing shutter between the opaque state of about five milliseconds and the transparent state at a second frequency of greater than about 50 hertz.

32. The method of claim 21, wherein the therapy sequence is adapted for a treatment of depression, and wherein the one or more properties of the visual system are utilized by alternately:
increasing transparency of the left and right crystal viewing shutters for a period of time; and
decreasing the transparency of the left and right crystal viewing shutters for the period of time.

33. The method of claim 21, wherein the therapy sequence is adapted for a treatment of reduced vision, and wherein the one or more properties of the visual system are utilized by alternately:
controlling the left crystal viewing shutter to be in an opaque state and the right crystal viewing shutter to be in a transparent state for a first time range of about 0.2 to 2 seconds; and
controlling the right crystal viewing shutter to be in the opaque state and the right crystal viewing shutter to be in the transparent state for a second time range of about 0.2 to 2 seconds.

34. The method of claim 21, wherein the therapy sequence is adapted for a treatment of strabismus by cycling one of the left and right crystal viewing shutters between an opaque state and a transparent state, the one of the left and right crystal viewing shutters being in the opaque state for about 20 seconds of a one minute time period.

35. The method of claim 21, wherein the therapy sequence is adapted for a treatment of attention deficit hyperactivity disorder by providing a repetitive signal using the left and right crystal viewing shutters over an extended period of time of at least one hour.

36. The method of claim 21, further comprising:
logging use times of the active glasses by the user; and
adjusting a duration of the therapy sequence based on the logged use times.

37. A computer readable program product stored on a tangible storage media for stimulating optic nerves, the program product when executed causing a computer processor to:
obtain a therapy sequence utilizing one or more properties of a visual system to stimulate the optic nerves of a user; and
perform a shutter activity based on the therapy sequence by:
controlling a left liquid crystal viewing shutter of active glasses; and
controlling a right liquid crystal viewing shutter of the active glasses.

38. The program product of claim 37, wherein the one or more properties of the visual system are selected from a group consisting of enhanced contrast, darkness adaptation, neighbor cell inhibition, pupil size modulation, and blink modulation.

39. The program product of claim 37, wherein the one or more properties of the visual system includes enhanced contrast, the enhanced contrast of the user being utilized by alternating each of the left and right crystal viewing shutters between an opaque state and a transparent state.

40. The program product of claim 39, wherein the left and right crystal viewing shutters are in the transparent state for no longer than 0.1 seconds.

41. The program product of claim 37, wherein the one or more properties of the visual system includes darkness adaptation, the darkness adaptation of the user being utilized by controlling one of the left and right crystal viewing stutters to be in an opaque state for at least 30 seconds.

42. The program product of claim 37, wherein the one or more properties of the visual system includes neighbor cell inhibition, the neighbor cell inhibition of the user being decreased by controlling each of the left and right crystal viewing stutters to be in an opaque state for at least 20 seconds for every two minute time period.

43. The program product of claim 37, wherein the one or more properties of the visual system includes pupil size modulation, the pupil size modulation of the user being utilized by intermittently alternating each of the left and right crystal viewing shutters between an opaque state and a transparent state.

44. The program product of claim 37, wherein the one or more properties of the visual system includes blink modulation, the blink modulation of the user being utilized by controlling at least one of the left and right crystal viewing stutters to be in an opaque state for a predetermined time that allows the user to perceive the opaque state as a nearby object.

45. The program product of claim 37, wherein the therapy sequence is adapted for a treatment of epilepsy, and wherein the one or more properties of the visual system are utilized by cycling the left crystal viewing shutter between an opaque state of less than about 0.1 seconds and a transparent state at a first frequency of less than about two hertz, the left crystal viewing shutter being in the opaque state for about one second of a four second time period.

46. The program product of claim 37, wherein the therapy sequence is adapted for a treatment of epilepsy, and wherein the one or more properties of the visual system are utilized by alternately:
cycling the left crystal viewing shutter between an opaque state of less than about 0.1 seconds and a transparent state at a first frequency of less than about two hertz, the left crystal viewing shutter being in the opaque state for about one second of a ten second time period; and
cycling the right crystal viewing shutter between the opaque state of less than about 0.1 seconds and the transparent state at a second frequency of less than about two hertz, the right crystal viewing shutter being in the opaque state for about one second of the ten second time period.

47. The program product of claim 37, wherein the therapy sequence is adapted for a treatment of epilepsy, and wherein the one or more properties of the visual system are utilized by alternately:
cycling the left crystal viewing shutter between an opaque state of about five milliseconds and a transparent state at a first frequency of greater than about 50 hertz; and
cycling the right crystal viewing shutter between the opaque state of about five milliseconds and the transparent state at a second frequency of greater than about 50 hertz.

48. The program product of claim 37, wherein the therapy sequence is adapted for a treatment of depression, and wherein the one or more properties of the visual system are utilized by alternately:
increasing transparency of the left and right crystal viewing shutters for a period of time; and
decreasing the transparency of the left and right crystal viewing shutters for the period of time.

49. The program product of claim 37, wherein the therapy sequence is adapted for a treatment of reduced vision, and wherein the one or more properties of the visual system are utilized by alternately:
controlling the left crystal viewing shutter to be in an opaque state and the right crystal viewing shutter to be in a transparent state for a first time range of about 0.2 to 2 seconds; and
controlling the right crystal viewing shutter to be in the opaque state and the right crystal viewing shutter to be in the transparent state for a second time range of about 0.2 to 2 seconds.

50. The program product of claim 37, wherein the therapy sequence is adapted for a treatment of strabismus by cycling one of the left and right crystal viewing shutters between an opaque state and a transparent state, the one of the left and right crystal viewing shutters being in the opaque state for about 20 seconds of a one minute time period.

51. The program product of claim 37, wherein the therapy sequence is adapted for a treatment of attention deficit hyperactivity disorder by providing a repetitive signal using the left and right crystal viewing shutters over an extended period of time of at least one hour.

52. The program product of claim 37, wherein the program product when executed further causes the computer processor to:
log use times of the active glasses by the user; and
adjust a duration of the therapy sequence based on the logged use times.

53. The system of claim 1, wherein one or more of the left and right liquid crystal viewing shutters comprise a pi cell.

54. The system of claim 1, wherein one or more of the left and right liquid crystal viewing shutters comprise a TN cell.

55. The system of claim 1, wherein the one or more properties of the visual system comprise enhanced contrast, darkness adaptation, neighbor cell inhibition, pupil size modulation, and blink modulation.
